(19) United States
(12) Patent Application Publication
(10) Pub. No.: US 2020/0197277 A1
(43) Pub. Date: Jun. 25, 2020

(54) COSMETIC AGENTS CONTAINING A COMBINATION OF AT LEAST TWO DIFFERENT ACTIVE INGREDIENTS

(71) Applicant: Henkel AG & Co. KGaA, Duesseldorf (DE)

(72) Inventors: INGA KERSTIN VOCKENROTH, Duesseldorf (DE); NATASCHA SCHEVARDO, Erkrath (DE); BERNHARD BANOWSKI, Duesseldorf (DE); RAINER SIMMERING, Grevenbroich (DE); HEIDE-MARIE HOEHNE, Koeln (DE)

(73) Assignee: Henkel AG & Co. KGaA, Duesseldorf (DE)

(21) Appl. No.: 16/706,010

(22) Filed: Dec. 6, 2019

(30) Foreign Application Priority Data
Dec. 19, 2018 (DE) 10 2018 222 233.9

(51) Int. Cl. A61K 8/41; A61K 8/34; A61K 8/49; A61K 8/362; A61K 8/46; A61Q 15/00; A61K 8/44

(52) U.S. Cl. CPC A61K 8/416; A61K 8/345; A61K 8/4926; A61K 8/44; A61K 8/466; A61K 8/4913; A61Q 15/00; A61K 8/362

(57) ABSTRACT

Cosmetic agents having a reduced amount of active ingredient, without adversely affecting the deodorizing effect, which also have excellent storage stability, contain in a cosmetically acceptable carrier ethylenediamine disuccinic acid and/or salts thereof and at least one benzalkonium chloride of the formula (I)

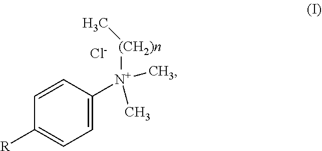

in which R stands for —H or -ethyl and n stands for an integer from the group of 7, 9, 11, 13, 15 or 17,
with the proviso that the total amount of compound(s) of formula (I) is from about 0.05 to about 5% by weight, based on the agent.

### CROSS-REFERENCE TO RELATED APPLICATION

This application claims priority to German Patent Application No. 10 2018 222 233.9, filed Dec. 19, 2018, which is incorporated herein by reference in its entirety.

### TECHNICAL FIELD

The present application relates to cosmetic agents containing a combination of at least two active ingredients. These cosmetic agents have an excellent deodorizing effect which also persists over a long period of time.

Furthermore, the present disclosure relates to a method for reducing and/or avoiding the body odor triggered by transpiration or perspiration, in which a cosmetic agent as contemplated herein is applied to the skin and remains there for at least one hour.

Finally, the present disclosure relates to the use of a cosmetic agent as contemplated herein for reducing and/or avoiding body odor triggered by transpiration.

### BACKGROUND

Eccrine and apocrine sweat glands are present in the human armpit. While the eccrine glands produce aqueous secretions in response to heat, the apocrine glands can secrete viscous secretions in response to stress. This apocrine sweat represents a complex mixture that contains steroids, cholesterol and other fats as well as about 10% protein. Unpleasant body odor under the armpit develops from the initially odorless secretion by development of bacterial decomposition of the ingredients of the apocrine sweat.

The decomposition products of apocrine sweat, which contribute significantly to the body odor, in particular the axillary body odor, can be divided into three classes: the first class form short-chain C₄-C₁₀ fatty acids, which can be linear, branched, saturated and unsaturated (for example isovaleric acid, 3-methyl-2-hexenoic acid, 3-hydroxy-3-methylhexanoic acid), the second class form short-chain linear or branched sulfanyl alcohols, and the third class from various steroid hormones and their metabolites (for example, 5-α-androstenol and 5-α-androstenone).

Body odor can therefore be combated by avoiding the bacterial degradation of sweat or by using perfume to cover the body odor. In order to avoid the bacterial degradation of perspiration, antimicrobial substances are used in the prior art which reduce the number of sweat-decomposing bacteria on the skin by killing or inhibiting the growth of these bacteria. Furthermore, active ingredients are known which reduce and/or prevent the formation of decomposition products by blocking bacterial enzymes. In addition, it is known to absorb the volatile decomposition products by physical and/or chemical interaction and to avoid unpleasant body odor in this way. In addition, the formation of body perspiration can be reduced by the use of antiperspirant active ingredients, so that fewer or no decomposition products can be formed by the sweat-decomposing bacteria. However, there is still a need for cosmetic agents which have both a high and a long-lasting deodorizing effect.

The use of benzalkonium chlorides in deodorants and as a deodorant active ingredient is widely described in the prior art, for example, in DE 10 2015 225 892 A1.

The incorporation of chelators or complexing agents in deodorants is disclosed in GB 2 422 780 A. EDTA, CDTA, EDDS, DTPA, TTHA and EDDHA are preferably used there to prevent the formation of fatty acids by bacteria on the skin.

Antimicrobial preparations, the benzalkonium chlorides and complexing agents from the group of EDTA (ethylenediaminetetraacetic acid), gluconate, GLDA (glutamic acid diacetic acid), EDDS (ethylenediamine-N,N′-disuccinic acid), DPTA (diethylenetriaminepentaacetic acid), HEDTA (hydroxyethylethylenediaminetriacetic acid), MGDA (methylglycinediacetic acid), PDTA (1,3-propylenediaminetetraacetic acid) and EDG (ethanoldiglycic acid) and mixtures thereof are disclosed in EP 2 797 413 Bl.

### BRIEF SUMMARY

Cosmetic agents and methods of using the same are provided. In an exemplary embodiment, a cosmetic agent includes components a) and b), wherein component b) is present in the cosmetic agent at from about 0.05 to about 5% by weight, based on a total weight of the cosmetic agent. Component a) is ethylenediamine disuccinic acid and/or salts thereof. Component b) is at least one benzalkonium chloride of formula (I),

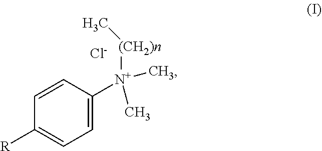

in which R stands for —H or -ethyl and n stands for an integer selected from the group of 7, 9, 11, 13, 15 or 17.

A method of preventing or reducing body odor is provided in another embodiment. The method includes the steps of applying a cosmetic agent to the skin, and leaving the cosmetic agent on the skin for at least 1 hour. The cosmetic agent includes components a) and b), wherein component b) is present in the cosmetic agent at from about 0.05 to about 5% by weight, based on a total weight of the cosmetic agent. Component a) is ethylenediamine disuccinic acid and/or salts thereof. Component b) is at least one benzalkonium chloride of formula (I)

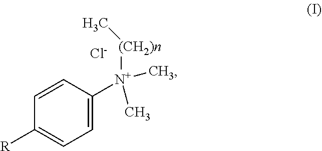

in which R stands for —H or -ethyl and n stands for an integer selected from the group of 7, 9, 11, 13, 15 or 17.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was to provide cosmetic agents which lead to a good and long-lasting action against body odor. Furthermore, the occurrence of skin inflammation and/or skin irritation during and/or after application of the cosmetic agent as contemplated herein should be avoided. In addition, the cosmetic agents should have a high storage stability and good cosmetic properties. The increased consumer interest in ecologically sound products should also be taken into account.

It has now surprisingly been found that not only a specific combating of odor-causing bacteria is achieved but also an unexpectedly long deodorizing effect is achieved by using a combination of benzalkonium chloride and EDDS. Due to the synergistic effect of the two aforementioned components of the cosmetic agents as contemplated herein, the amount of active ingredients can be reduced without adversely affecting the deodorizing effect. Due to the reduced amount of active ingredients, the cosmetic agents as contemplated herein cause little irritation and sensitizing. Furthermore, the use of the active ingredient combination does not lead to a negative impact on the storage stability of the cosmetic agents as contemplated herein, so that these agents have excellent storage stability. Finally, these agents have good cosmetic properties.

A first subject of the present application is therefore a cosmetic agent, containing in a cosmetically acceptable carrier

a) ethylenediamine disuccinic acid and/or salts thereof; and
b) at least one benzalkonium chloride of the formula (I)

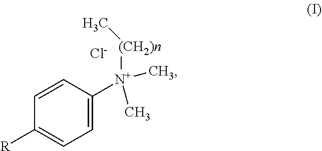

in which R stands for —H or -ethyl and n stands for an integer from the group of 7, 9, 11, 13, 15 or 17,
with the proviso that the total amount of compound(s) of formula (I) is from about 0.05 to about 5% by weight, based on the agent.

The use of a combination of ethylenediamine disuccinic acid and/or salts thereof with a benzalkonium chloride of the formula (I) achieves an excellent reduction in the body odor triggered by transpiration, in particular under the armpit, and especially under the armpit. In addition, it has been found that the above-mentioned combination of the two components a) and b) of the cosmetic agents as contemplated herein has a synergistic increase in deodorizing performance, so that the amount of active ingredients can be reduced without adversely affecting the deodorizing effect. The cosmetic agents as contemplated herein have an improved skin tolerance due to the reduced amount of active ingredients. In addition, the cosmetic properties of the cosmetic agents as contemplated herein can be improved by using the above-mentioned combination of active ingredients. Finally, the use of components a) and b) does not have a negative influence on the storage stability of the cosmetic agents as contemplated herein.

The term “fatty acid” in the context of the present disclosure is understood to mean aliphatic carboxylic acids which have unbranched or branched carbon radicals having 4 to about 40 carbon atoms. The fatty acids used in the context of the present disclosure can be both naturally occurring and synthetically produced fatty acids. Furthermore, the fatty acids can be monounsaturated or polyunsaturated.

The specification % by weight refers in the present case, unless stated otherwise, to the total weight of the cosmetic agents as contemplated herein, wherein the sum of all ingredients of the agents as contemplated herein results in 100% by weight. Furthermore, the statement % by weight, unless otherwise specified, refers to the amount of the respective component in the propellant-free cosmetic agent, so that the amount of optionally present propellant is not taken into account in the calculation of the total weight of the cosmetic agents.

The cosmetic agent contains constituents a) and b) in a cosmetically acceptable carrier. This preferably contains at least one component selected from water, a C₁-C₄ alcohol, a cosmetic oil that is liquid under normal conditions and mixtures thereof. The cosmetic oils that are liquid under normal conditions are immiscible with water and are neither fragrances nor essential oils. In the context of the present application, “normal conditions” are a temperature of about 20° C. and a pressure of about 1013 hPa.

Suitable cosmetic carriers are, for example, aqueous or aqueous-alcoholic carriers. These are understood to mean carriers which contain more than about 5.0% by weight of water or more than about 5.0% by weight of water and at least one C₁-C₄ alcohol, in each case based on the total weight of the cosmetic agent. Aqueous carriers preferably contain free water in a total amount of from about 10 to about 96% by weight, preferably from about 15 to about 80% by weight, more preferably from about 30 to about 70% by weight, in particular from about 40 to about 60% by weight, based on the total weight of the cosmetic agent. In aqueous-alcoholic carriers, ethanol is preferably present in a total amount of from about 0.1 to about 50% by weight, preferably from about 0.5 to about 30% by weight, more preferably from about 1.0 to about 20% by weight, in particular from about 1.0 to about 9.0% by weight, based on the total weight of the cosmetic agent.

The abovementioned aqueous and aqueous-alcoholic carriers can additionally contain at least one (C₂-C₆) alkyl alcohol having 2 or 3 hydroxyl groups, in particular 1,2-propylene glycol, 1,3-propylene glycol, glycerol and 1,3-butylene glycol.

However, it can also be provided to use an anhydrous cosmetic carrier. Anhydrous carriers are understood as contemplated herein to mean carriers which contain less than about 5.0% by weight, preferably less than about 4.0% by weight, more preferably less than about 3.0% by weight, in particular 0% by weight of free water, based on the total weight of the cosmetic agent. In the context of the present disclosure, free water is understood to mean water which is different from water of crystallization, water of hydration or similar molecularly bound water of the constituents used. When calculating the total amount of free water, however, water of crystallization, water of hydration or similar molecularly bound water of the constituents used are disregarded.

In this context, in particular alcoholic media are used as a cosmetically acceptable medium. It is possible in particular to include the lower alcohols having 1 to 4 carbon atoms, for example, ethanol and isopropanol, which are customarily used for cosmetic purposes as alcohols. Alcoholic carriers preferably contain ethanol in a total amount of from about 20 to about 95% by weight, preferably from about 25 to about 65% by weight, in particular from about 25 to about 60% by weight, based on the total weight of the cosmetic agent.

Furthermore, it is particularly preferred in this context to use liquid cosmetic oils as a cosmetic carrier. These cosmetic oils can be selected from the group of (i) volatile non-silicone oils, in particular liquid paraffin oils and isoparaffin oils such as isodecane, isoundecane, isododecane, isotridecane, isotetradecane, isopentadecane, isohexadecane and isoeicosan; (ii) non-volatile non-silicone oils, in particular the esters of linear or branched saturated or unsaturated C₂₋₃₀ fatty alcohols with linear or branched saturated or unsaturated C₂₋₃₀ fatty acids which can be hydroxylated, the C₈-C₂₂ fatty alcohol esters of monovalent or polyvalent C₂-C₇ hydroxycarboxylic acids, the dicarboxylic acid esters of linear or branched C₂-C₁₀ alkanols, the addition products of ethylene oxide and/or propylene oxide to mono- or polyvalent C₃₋₂₂ alkanols, which can optionally be esterified, the symmetrical, unsymmetrical or cyclic esters of carbonic acid with fatty alcohols, the esters of dimers of unsaturated C₁₂₋₂₂ fatty acids with monovalent, linear, branched and cyclic C₂₋₁₈ alkanols or C₂₋₆ alkanols, the benzoic acid esters of linear or branched C₈₋₂₂ alkanols such as benzoic acid C₁₂₋₁₅ alkyl ester and benzoic acid isostearyl ester and benzoic acid octyldodecyl ester, the synthetic hydrocarbons, such as polyisobutene and polydecenes, the alicyclic hydrocarbons; and (iii) mixtures thereof.

The term “volatile cosmetic oil” refers, as contemplated herein, to cosmetic oils which, at about 20° C. and an ambient pressure of about 1013 hPa, have a vapor pressure from about 2.66 Pa to about 40,000 Pa (from about 0.02 to about 300 mm Hg), preferably from about 10 to about 12,000 Pa (from about 0.1 to about 90 mm Hg), more preferably from about 13 to about 3000 Pa (from about 0.1 to about 23 mm Hg), in particular from about 15 to about 500 Pa (from about 0.1 to about 4 mm Hg). In addition, the term “nonvolatile cosmetic oils” in the context of the present disclosure is understood to mean cosmetic oils which have a vapor pressure of less than about 2.66 Pa (about 0.02 mm Hg) at about 20° C. and an ambient pressure of about 1013 hPa.

The use of mixtures of the abovementioned cosmetic oils, in particular of nonvolatile and volatile cosmetic oils, is furthermore preferred as contemplated herein since parameters such as skin feel, visibility of the residue and stability of the cosmetic agent as contemplated herein can be set and the agent can thus be better adapted to the needs of the consumer.

In the context of the present disclosure, it is preferred when the cosmetic oil which is liquid at about 20° C. and about 1013 hPa is used in a total amount from about 1.0 to about 98% by weight, preferably from about 2.0 to about 95% by weight, more preferably from about 5.0 to about 70% by weight, yet more preferably from about 10 to about 60% by weight, in particular from about 15 to about 50% by weight, based on the total weight of the cosmetic agent.

As a first essential constituent a), the cosmetic agent as contemplated herein contains ethylenediamine disuccinic acid and/or salts thereof:

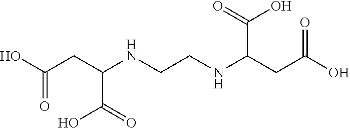 [SMILES: O=C(O)CC(NCCNC(CC(=O)O)C(=O)O)C(=O)O]

The four acid groups can be partially or completely neutralized, wherein alkali metal salts and, in particular, sodium salts are preferably used. The trisodium salt

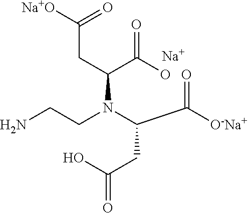

is particularly preferred.

Regardless of whether the free acid and/or its salts are used, cosmetic agents as contemplated herein are preferred which contain, based on their weight, from about 0.01 to about 1% by weight, preferably from about 0.02 to about 0.8% by weight, more preferably from about 0.03 to about 0.7% by weight, particularly preferably from about 0.04 to about 0.6% by weight and in particular from about 0.05 to about 0.5% by weight of ethylenediamine disuccinic acid and/or salts thereof.

The percentages by weight specifications relate to the total amount of EDDS and salt(s) of EDDS. Thus, if mixtures of free acid and various salts of EDDS are used, the total amount of all these substances is most preferably from about 0.05 to about 0.5% by weight.

Preferably, no further complexing agents are used in addition to the EDDS or its salts. In particular, the use of EDTA should be omitted so as not to impair the performance of the combination as contemplated herein. Preferred cosmetic agents as contemplated herein are free of EDTA and/or its salts.

As a second essential constituent b), the cosmetic agent as contemplated herein contains at least one benzalkonium chloride of the formula (I)

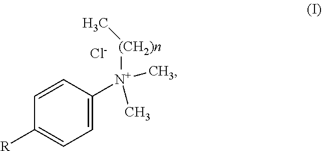

in which R stands for —H or -ethyl and n stands for an integer from the group of 7, 9, 11, 13, 15 or 17,
with the proviso that the total amount of compound(s) of formula (I) is from about 0.05 to about 5% by weight, based on the agent.

Preferred agents as contemplated herein use benzalkonium chlorides, in which R stands for —H. Very particular preference is given to the compounds lauryldimethylbenzalkonium chloride (R=—H, n=11), myristyldimethylbenzalkonium chloride (R=—H, n=13), cetyldimethylbenzalkonium chloride (R=—H, n=15) and/or stearyldimethylbenzalkonium chloride (R=—H, n=17).

Regardless of whether a pure compound or mixtures of different compounds which fall under formula (I) are used, cosmetic agents as contemplated herein are preferred which, based on their weight, contain from about 0.05 to about 2.5% by weight, preferably from about 0.075 to about 2% by weight, more preferably from about 0.1 to about 1% by weight, particularly preferably from about 0.15 to about 0.75% by weight and in particular from about 0.2 to about 0.5% by weight of benzalkonium chloride(s) of formula (I).

Preferably, the agents as contemplated herein are formulated so that the total amount of ingredients a) and b) is within certain ranges of amounts. It has been shown that the antibacterial effect, in particular the effect against S. epidermidis, S. hominis, C. tuberculostearicum and A. octavius, is particularly pronounced within these ranges of amounts. Particularly effective and thus preferred cosmetic agents as contemplated herein include a total amount of ingredients a) and b), based on the weight of the agent, of from about 0.075 to about 2.5% by weight, preferably from about 0.1 to about 2% by weight, more preferably from about 0.125 to about 1.5% by weight, particularly preferably from about 0.15 to about 1.0% by weight and in particular from about 0.2 to about 0.5% by weight.

A further increase in effectiveness can be observed when the two ingredients a) and b) are in specific weight ratios to each other. Here, particularly effective and therefore preferred cosmetic agents as contemplated herein are exemplified in that the weight ratio of the ingredients a) and b) to each other is from about 5:1 to about 1:5, preferably from about 3:1 to about 1:3, more preferably from about 2:1 to about 1:2, even more preferably from about 3:2 to about 2:3 and in particular from about 12:9 to about 1:1.

The cosmetic agents as contemplated herein can contain further deodorizing and/or transpiration inhibiting substances. Preferred cosmetic agents as contemplated herein additionally contain one or more active ingredients selected from the group of triethyl citrate, 3-[(2-ethylhexyl) oxy]-1,2-propanediol, 2-benzylheptanol, phenoxyethanol, cocamidopropyl PG-dimonium chloride phosphate, tropolone, silver lactate, magnesium oxide, aluminum chlorohydrate, aluminum-zirconium tetrachlorohydrex glycine and mixtures thereof.

The cosmetic agents as contemplated herein can contain certain aluminum salts. It is therefore preferred in the context of the present disclosure when the cosmetic agent contains at least one aluminum salt selected from the group of (i) water-soluble inorganic salts of aluminum, in particular aluminum chlorohydrate, aluminum sesquichlorohydrate, aluminum dichlorohydrate, aluminum hydroxide, potassium aluminum sulfate, aluminum bromohydrate, aluminum chloride, aluminum sulfate; (ii) water-soluble organic salts of aluminum, in particular aluminum chlorohydrex-propylene glycol, aluminum chlorohydrex-polyethylene glycol, aluminum-propylene glycol complexes, aluminum sesquichlorohydrex-propylene glycol, aluminum sesquichlorohydrex-polyethylene glycol, aluminum-propylene glycol-dichlorhydrex, aluminum-polyethylene glycol-dichlorhydrex, aluminum undecylenoyl collagen amino acid, sodium aluminum lactate, sodium aluminum chlorhydroxylactate, aluminum lipoamino acids, aluminum lactate, aluminum chlorohydroxyallantoinate, sodium aluminum chlorohydroxylactate; and (iii) mixtures thereof.

Furthermore, the cosmetic agents as contemplated herein can contain special aluminum-zirconium salts. Preferred embodiments of the cosmetic agent contain at least one aluminum-zirconium salt selected from the group of (i) water-soluble inorganic aluminum-zirconium salts, in particular aluminum-zirconium trichlorohydrate, aluminum-zirconium tetrachlorohydrate, aluminum-zirconium pentachlorohydrate, aluminum-zirconium octachlorohydrate; (ii) water-soluble organic aluminum-zirconium salts, in particular aluminum-zirconium-propylene glycol complexes, aluminum-zirconium trichlorohydrex glycine, aluminum-zirconium tetrachlorohydrex glycine, aluminum-zirconium pentachlorohydrex glycine, aluminum-zirconium octachlorohydrex glycine; and (iii) mixtures thereof.

The terms “aluminum salts” and “aluminum-zirconium salts” are understood to mean, as contemplated herein, no aluminosilicates and zeolites. Furthermore, as contemplated herein, water-soluble aluminum salts or water-soluble aluminum-zirconium salts are understood to mean those salts which have a solubility of at least about 3% by weight at about 20° C., that is, at least about 3 g of the antiperspirant aluminum salt or aluminum-zirconium salt dissolve in about 97 g water at about 20° C.

Especially preferred inorganic aluminum salts are selected from aluminum chlorohydrate, in particular aluminum chlorohydrate having the general formula [Al₂(OH)₅Cl.1-6H₂O]ₙ, more preferably [Al₂(OH)₅Cl.2-3H₂O]ₙ, which can be present in unactivated (polymerized) or in active (depolymerized) form, and aluminum chlorohydrate having the general formula [Al₂(OH)₄Cl₂.1-6H₂O]ₙ, preferably [Al₂(OH)₄Cl₂.2-3H₂O]ₙ, which can be present in unactivated (polymerized) or in active (depolymerized) form.

As contemplated herein, particularly preferred antiperspirant aluminum salts are selected from so-called “activated” aluminum salts, which are also referred to as “antiperspirant active ingredients” having enhanced activity. Activated aluminum salts are typically produced by heat treating a dilute solution of the corresponding salt (for example, a solution having about 10% by weight of salt) to increase its HPLC peak 4-to-peak 3 area ratio. The activated salt can then be dried to a powder, in particular spray-dried. In addition to spray drying, for example, roller drying is also suitable. Activated aluminum salts typically have a HPLC peak 4-to-peak 3 area ratio of at least about 0.4, preferably at least about 0.7, in particular at least about 0.9, wherein at least about 70% of the aluminum are attributable to these HPLC peaks.

Also known in this context are “activated” aluminum-zirconium salts which have a high HPLC peak 5 aluminum content, in particular a peak 5 area of at least about 33%, preferably of at least about 45%, based on the total area under the peaks 2 to 5, measured by HPLC of a about 10% by weight aqueous solution of the active ingredient under conditions in which the aluminum species are resolved into at least 4 consecutive peaks (designated with peaks 2 to 5). Preferred aluminum-zirconium salts having a high HPLC peak 5 aluminum content are also referred to as “E⁵AZCH”. Furthermore, the above-mentioned activated aluminum-zirconium salt can be additionally stabilized with a water-soluble strontium salt and/or with a water-soluble calcium salt.

It is also possible as contemplated herein to use antiperspirant aluminum salts as nonaqueous solutions or solubilizates of an activated antiperspirant aluminum or aluminum-zirconium salt. Such aluminum or aluminum-zirconium salts are stabilized by the addition of an effective amount of a polyvalent alcohol having 3 to 6 carbon atoms and 3 to 6 hydroxyl groups, preferably propylene glycol, sorbitol and pentaerythritol, against the loss of activation of the salt.

Also particularly preferred are complexes of activated antiperspirant aluminum or aluminum-zirconium salts with a polyvalent alcohol containing from about 20 to about 50% by weight, preferably from about 20 to about 42% by weight of activated antiperspirant aluminum or aluminum-zirconium salt and from about 2 to about 16% by weight of molecularly bound water, wherein the remainder is to 100% by weight of at least one polyvalent alcohol having 3 to 6 carbon atoms and 3 to 6 hydroxyl groups. Propylene glycol, propylene glycol/sorbitol mixtures and propylene glycol/pentaerythritol mixtures are such preferred alcohols.

In the context of the present disclosure, it is likewise possible to use basic calcium aluminum salts as antiperspirant aluminum salts. These salts can be obtained by reacting calcium carbonate with aluminum chlorhydroxide or aluminum chloride and aluminum powder or by adding calcium chloride dihydrate to aluminum chlorhydroxide. However, aluminum-zirconium complexes are also possible which are buffered with salts of amino acids, in particular with alkali and alkaline earth glycinates.

Aluminum or aluminum-zirconium salts which are preferably stabilized by amino acids, in particular glycine, hydroxyalkanoic acids, in particular glycolic acid and lactic acid, or betaines, can also be used as the antiperspirant activated aluminum and aluminum-zirconium salts preferred as contemplated herein.

Further preferred activated aluminum salts are those of the general formula Al₂(OH)₆₋ₐXa, wherein X stands for Cl, Br, I or NO₃ and “a” stands for a number from about 0.3 to about 5, preferably from about 0.8 to about 2.5, and in particular from about 1 to about 2, so that the molar ratio of Al:X is from about 0.9:1 to about 2.1:1. Particularly preferred is aluminum chlorohydrate (that is, X in the aforementioned formula stands for Cl) and especially 5/6 basic aluminum chlorohydrate with “a”=1, so that the molar ratio of aluminum to chlorine is from about 1.9:1 to about 2.1:1.

Preferred activated aluminum-zirconium salts are those of the general formula ZrO(OH)_{2-pb}Y_{b}, wherein Y stands for Cl, Br, I, NO₃ or SO₄, b stands for a rational number from about 0.8 to about 2 and p stands for the valence of Y such that the Al:Zr molar ratio is from about 2 to about 10 and the metal:(X+Y) ratio is from about 0.73 to about 2.1, preferably from about 0.9 to about 1.5. A particularly preferred salt is aluminum-zirconium chlorohydrate (that is, X and Y stand for Cl), which has an Al:Zr ratio of from about 2 to about 10 and a molar metal:Cl ratio of from about 0.9 to about 2.1.

Particularly preferred antiperspirant aluminum salts as contemplated herein have a molar metal to chloride ratio of from about 1.9 to about 2.1. The metal to chloride ratio of aluminum sesquichlorohydrates which is likewise particularly preferred in the context of the present disclosure is from about 1.5:1 to about 1.8:1. Preferred aluminum-zirconium tetrachlorohydrates have a molar ratio of Al:Zr from about 2 to about 6 and of metal:chloride from about 0.9 to about 1.3, wherein in particular salts having a molar metal to chloride ratio of from about 0.9 to about 1.1, preferably from about 0.9 to about 1.0 are preferred. In the context of the present disclosure, it is preferred when certain of the aforementioned active ingredients are used. Preferred embodiments of the present disclosure are a cosmetic agent that contains at least one first active ingredient selected from the group of triethyl citrate, 3-[(2-ethylhexyl)oxy]-1,2-propanediol, 2-benzylheptanol, phenoxyethanol, cocamidopropyl PG-dimonium chloride phosphate, 1,2-hexanediol, 1,2-octanediol, 1,2-decanediol, tropolone, silver lactate, magnesium oxide, aluminum chlorohydrate, aluminum-zirconium tetrachlorohydrexglycine, and mixtures thereof. The use of these agents has proven to be particularly advantageous in terms of deodorizing and/or antiperspirant performance. In particular, a synergistic increase in deodorant and/or antiperspirant performance is achieved. In addition, both the skin compatibility and the cosmetic properties of the agents as contemplated herein can be further increased by the use of these active ingredients. These compounds have the following chemical names and CAS numbers:

| Preservative | Chemical name/structural formula | CAS number |
|---|---|---|
| Triethylcitrate | 2-Hydroxypropane-1,2,3-tricarboxylic | 77-93-0 |
| | acid triethyl ester | |
| 3-[(2-ethylhexyl)oxy]- | OCTOXYGLYCEROL | 70445-33-9 |
| 1,2-propanediol | | |
| 2-benzylheptanol | 2-benzyl-1-heptanol | 92368-90-6 |
| Phenoxyethanol | 2-phenoxy-1-ethanol | 122-99-6 |
| Cocamidopropyl PG- | Propanaminium, 2,3-dihydroxy-N,N- | 83682-78-4 |
| dimonium chloride | dimethyl-N-(coconut-alkyl)-, 3-sodium | |
| phosphate | hydrogen phosphate ester, chloride | |
| 1,2-hexanediol | CH₃(CH₂)₃CH(OH)CH₂OH | 6920-22-5 |
| 1,2-octanediol | CH₃(CH₂)₅CH(OH)CH₂OH | 1117-86-8 |
| 1,2-decanediol | CH₃(CH2)₇CH(OH)CH₂OH | 1119-86-4 |
| Tropolone | 2-hydroxy-2,4,6-cycloheptatrien-1-one | 38768-08-0 |
| Silver lactate | Silver lactate | 128-00-7 |
| Magnesium oxide | Magnesium oxide, MgO | 1309-48-4 |
| Aluminum chlorohydrate | Aluminum chloride, basic | 1327-41-9 |
| Aluminum-zirconium | Al₄Zr(OH)₁₂Cl₄Gly × nH₂O | 90604-80-1 |
| tetrachlorohydrex | | |
| glycine | | |

The deodorizing and/or antiperspirant effect can be further increased when the cosmetic agents contain more than one of the previously cited active ingredients. Preferred cosmetic agents are therefore exemplified by having at least two active ingredients selected from the group of triethyl citrate, 3-[(2-ethylhexyl)oxy]-1,2-propanediol, 2-benzylheptanol, phenoxyethanol, cocamidopropyl PG-dimonium chloride phosphate, 1,2-hexanediol, 1,2-octanediol, 1,2-decanediol, tropolone, silver lactate, magnesium oxide, aluminum chlorohydrate, aluminum-zirconium tetrachlorohydrexglycine, and mixtures thereof.

Further preferred cosmetic agents as contemplated herein contain at least three active ingredients selected from the group of triethyl citrate, 3-[(2-ethylhexyl)oxy]-1,2-propanediol, 2-benzylheptanol, phenoxyethanol, cocamidopropyl PG-dimonium chloride phosphate, 1,2-hexanediol, 1,2-octanediol, 1,2-decanediol, tropolone, silver lactate, magnesium oxide, aluminum chlorohydrate, aluminum-zirconium tetrachlorohydrexglycine, and mixtures thereof.

The cosmetic agents as contemplated herein preferably contain the above-mentioned active ingredient(s) in certain ranges of amounts. Preferred cosmetic agents as contemplated herein contain, based on their total weight, of from about 0.0005 to about 35% by weight, preferably from about 0.005 to about 25% by weight, more preferably from about 0.01 to about 10% by weight, in particular from about 0.05 to about 8.0% by weight, of at least one active ingredient selected from the group of triethyl citrate, 3-[(2-ethylhexyl)oxy]-1,2-propanediol, 2-benzylheptanol, phenoxyethanol, cocamidopropyl PG-dimonium chloride phosphate, 1,2-hexanediol, 1,2-octanediol, 1,2-decanediol, tropolone, silver lactate, magnesium oxide, aluminum chlorohydrate, aluminum-zirconium tetrachlorohydrexglycine, and mixtures thereof. The use of such amounts of the at least one active ingredient leads to a synergistic increase in the deodorizing and/or antiperspirant performance. As a result, the amount of active ingredients used can be reduced without adversely affecting the deodorizing and/or antiperspirant performance. The cosmetic agents as contemplated herein are therefore particularly kind to the skin.

Due to the powerful combination as contemplated herein, the agents as contemplated herein can also be formulated without loss of performance without the use of aluminum salts. Usually, the effectiveness against body odor in aluminum-free products is comparatively low, wherein the performance deficit makes itself noticeable in particular after from about 24 to about 48 hours. The combination as contemplated herein achieves a significant improvement in the deodorant performance over a period of from about 24 to about 48 hours after administration.

Preferred cosmetic agents as contemplated herein are free of aluminum salts.

As a further constituent, the cosmetic agents as contemplated herein can contain at least one active ingredient selected from the group of cetylpyridinium chloride, zinc hydroxide carbonate, zinc phenolsulfonate, polyglyceryl-2-caprate, sorbitan caprylate, octenidine, charnesolic acid, tartaric acid, and mixtures thereof. Here, preferred cosmetic agents as contemplated herein additionally contain one or more active ingredients selected from the group of cetylpyridinium chloride, zinc hydroxide carbonate, zinc phenolsulfonate, polyglyceryl-2-caprate, sorbitan caprylate, octenidine, charnesolic acid, tartaric acid, and mixtures thereof.

In the context of the present disclosure, it is preferred when certain active ingredients are used. Preferred embodiments of the present disclosure are a cosmetic agent that additionally contains one or more active ingredients selected from the group of cetylpyridinium chloride, zinc hydroxide carbonate, zinc phenolsulfonate, polyglyceryl-2-caprate, sorbitan caprylate, octenidine, charnesolic acid, tartaric acid, and mixtures thereof.

These compounds have the following chemical names and CAS numbers:

| Preservative | Chemical name/structural formula | CAS number |
|---|---|---|
| Cetylpyridinium chloride | 1-hexadecylpyridinium chloride | 123-03-5 |
| Zinc hydroxide carbonate | [ZnCO₃]₂*[Zn(OH)₂]₃ | 5263-02-5 |
| Zinc phenolsulfonate | Zinc phenolsulfonate | 127-82-2 |
| Polyglyceryl-2 caprate | 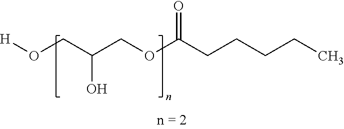 | 156153-06-9 |
| Sorbitan caprylate | Sorbitan mono octanoate | 60177-36-8 |
| Octenidine | N-Octyl-1-[10-(4-octyliminopyridine-1- | 70775-75-6 |
| | yl)decyl]pyridine-4-imine dihydrochloride | |
| Charnesolic acid | (4aR, 10aS)-5,6-dihydroxy-1,1-dimethyl-7- | 3650-09-7 |
| | propan-2-yl-2,3,4,9,10,10a- | |
| | hexahydrophenanthren-4a-carboxylic acid | |
| Tartaric acid | D- and/or L-tartaric acid | 133-37-9 |

The deodorizing and/or antiperspirant effect can be further increased when the cosmetic agents contain more than one of the previously cited active ingredients. Preferred cosmetic agents therefore contain at least two active ingredients selected from the group of cetylpyridinium chloride, zinc hydroxide carbonate, zinc phenolsulfonate, polyglyceryl-2-caprate, sorbitan caprylate, octenidine, charnesolic acid, tartaric acid, and mixtures thereof.

Further preferred cosmetic agents as contemplated herein contain at least three active ingredients selected from the group of cetylpyridinium chloride, zinc hydroxide carbonate, zinc phenolsulfonate, polyglyceryl-2-caprate, sorbitan caprylate, octenidine, charnesolic acid, tartaric acid, and mixtures thereof.

The cosmetic agents as contemplated herein preferably contain the second active ingredient in certain ranges of amounts. Preferred cosmetic agents as contemplated herein contain, based on their total weight, from about 0.001 to about 20% by weight, preferably from about 0.005 to about 15% by weight, more preferably from about 0.01 to about 10% by weight, in particular from about 0.05 to about 2.0% by weight, of at least one further active ingredient selected from the group of cetylpyridinium chloride, zinc hydroxide carbonate, zinc phenolsulfonate, polyglyceryl-2-caprate, sorbitan acylate, octenidine, charnesolic acid, tartaric acid, and mixtures thereof.

The cosmetic agents as contemplated herein can additionally contain additional deodorant active ingredients (C). However, the additional deodorant active ingredient (C) is different from the first active ingredient (A) and the second active ingredient (B).

It can be preferred within the context of the present disclosure when the cosmetic agent additionally contains a deodorant active ingredient (C). Preferred agents additionally contain at least one deodorant active ingredient (C) selected from the group of (i) active ingredients against exoesterases, in particular against arylsulfatase, lipase, beta-glucuronidase and cystathion-β-lyase; (ii) odor absorbers, in particular silicates, such as montmorillonite, kaolinite, illite, beidellite, nontronite, saponite, hectorite, bentonite, smectite and talcum, zeolites, zinc ricinoleate, cyclodextrins; (iii) deodorizing ion exchangers; (iv) antimicrobial agents; (v) prebiotic active components; and (vi) mixtures thereof.

Silicates serve as odor absorbers, which can simultaneously advantageously support the rheological properties of the cosmetic agent as contemplated herein. Among the silicates which are particularly advantageous as contemplated herein are, especially, phyllosilicates and, among these, in particular montmorillonite, kaolinite, illite, beidellite, nontronite, saponite, hectorite, bentonite, smectite, and talcum. Further preferred odor absorbers are, for example, zeolites, zinc ricinoleate, cyclodextrins, certain metal oxides, such as alumina, and chlorophyll.

Furthermore, the deodorant active can be selected from the group of terpene alcohols such as farnesol, chlorophyllin copper complexes, carboxylic acid esters, in particular carboxylic acid monoesters of mono-, di- and triglycerol (in particular glycerol monolaurate, diglycerol monocaprate, diglycerol monolaurate, triglycerol monolaurate and triglycerol monomyristate) and plant extracts (for example, green tea and constituents of linden flower oil).

Further preferred deodorant active ingredients are selected from so-called prebiotically active components, which as contemplated herein are to be understood to mean those components which inhibit only or at least predominantly the odor-causing germs of the skin microflora, but not the desired ones, that is, the non-odor-forming germs. Explicitly named here are active ingredients such as conifer extracts, in particular from the group of Pinaceae, and plant extracts from the group of Sapindaceae, Araliaceae, Lamiaceae and Saxifragaceae, in particular extracts of Picea spp., Paullinia sp., Panax sp., Lamium album or Ribes nigrum and mixtures to name these substances.

Further preferred deodorant active ingredients are selected from the germ-inhibiting perfume oils and the Deosafe® perfume oils, which are available from the company Symrise, formerly Haarmann and Reimer.

The enzyme inhibitors include substances which inhibit the enzymes responsible for the sweat decomposition, in particular arylsulfatase, β-glucuronidase, aminoacylase, ester-splitting lipases and lipoxygenase, for example, zinc glycinate.

The at least one further deodorant active ingredient (C) is preferably used in the cosmetic agents as contemplated herein in certain ranges of amounts. It is therefore preferred in this context when they contain, based on their total weight, from about 0.005 to about 20% by weight, preferably from about 0.1 to about 15% by weight, more preferably from about 0.1 to about 13% by weight, in particular of from about 0.1 to about 10% by weight of at least one deodorant active ingredient (C). If a mixture of deodorant active ingredients is used, the amounts cited above refer to the mixture of these active ingredients.

In the context of the present disclosure, it can be provided that the cosmetic agents as contemplated herein, in addition to the additional deodorant active ingredients cited above, contain further ingredients selected from the group including (i) waxes; (ii) emulsifiers and/or surfactants; (iii) hydrogel formers; (iv) skin-cooling active ingredients; (v) propellants; (vi) thickening agents and (vii) mixtures thereof.

The cosmetic agents as contemplated herein can additionally contain at least one wax. In the context of the present disclosure, the term “waxes” is understood to mean substances which are kneadable or solid to brittle at about 20° C., have a coarse to fine crystalline structure and are translucent to opaque in color but not glassy. Furthermore, these substances melt above about 25° C. without decomposition, are slightly liquid (less viscous) above the melting point, have a strong temperature-dependent consistency and solubility and are polishable under light pressure. It is therefore advantageous as contemplated herein when the cosmetic agent additionally contains at least one wax selected from the group of (i) coconut fatty acid glycerol mono-, di- and triesters; (ii) Butyrospermum parkii (shea butter); (iii) esters of saturated monovalent C₈₋₁₈ alcohols with saturated C₁₂₋₁₈ monocarboxylic acids; (iv) linear, primary C₁₂-C₂₄ alkanols; (v) esters of a saturated, monovalent C₁₆-C₆₀ alkanol and a saturated C₈-C₃₆ monocarboxylic acid, in particular cetyl behenate, stearyl behenate and C₂₀-C₄₀ alkyl stearate; (vi) glycerol triesters of saturated linear C₁₂-C₃₀ carboxylic acids which can be hydroxylated, in particular hydrogenated palm oil, hydrogenated coconut oil, hydrogenated castor oil, glyceryl tribehenate and glyceryl tri-12-hydroxy stearate; (vii) natural vegetable waxes, in particular candelilla wax, carnauba wax, Japan wax, sugarcane wax, ouricoury wax, cork wax, sunflower wax, fruit waxes; (viii) animal waxes, in particular beeswax, shellac wax and spermaceti; (ix) synthetic waxes, in particular montan ester waxes, hydrogenated jojoba waxes and sasol waxes, polyalkylene waxes and polyethylene glycol waxes, C₂₀-C₄₀ dialkyl esters of dimer acids, C₃₀₋₅₀ alkyl beeswax and alkyl and alkylaryl esters of dimer fatty acids, paraffin waxes; and (x) mixtures thereof. The cosmetic agent contains the additional at least one wax preferably in a total amount of from about 0.01 to about 20% by weight, preferably from about 3 to about 20% by weight, more preferably from about 5 to about 18% by weight, in particular from about 6 to about 15% by weight, based on the total weight of the cosmetic agent.

Emulsifiers and surfactants which are preferably suitable as contemplated herein are selected from anionic, cationic, nonionic, amphoteric, in particular ampholytic and zwitterionic emulsifiers and surfactants. Surfactants are amphiphilic (bifunctional) compounds which include at least one hydrophobic and at least one hydrophilic molecule part. The hydrophobic radical is preferably a hydrocarbon chain having 8 to about 28 carbon atoms, which can be saturated or unsaturated, linear or branched. Particularly preferably, this C₈-C₂₈ alkyl chain is linear.

Anionic surfactants are understood to mean surfactants with exclusively anionic charges; they contain, for example, carboxyl groups, sulfonic acid groups or sulfate groups. Particularly preferred anionic surfactants are alkyl sulfates, alkyl ether sulfates, acyl glutamates and C₈₋₂₄ carboxylic acids and their salts, the so-called soaps.

Cationic surfactants are understood to mean surfactants with exclusively cationic charges; they contain, for example, quaternary ammonium groups. Preference is given to cationic surfactants of the type of quaternary ammonium compounds, esterquats and amidoamines Preferred quaternary ammonium compounds are ammonium halides and the imidazolium compounds known under the INCI names Quaternium-27 and Quaternium-83. Further cationic surfactants which can be used as contemplated herein represent the quaternized protein hydrolyzates. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines.

The amphoteric surfactants are subdivided into ampholytic surfactants and zwitterionic surfactants. Ampholytic surfactants are understood to mean those surface-active compounds which have both acidic (for example —COOH or —SO₃H groups) and basic hydrophilic groups (for example amino groups) and thus behave acidic or basic depending on the condition. Zwitterionic surfactants are understood by the person skilled in the art to be surfactants which carry both a negative and a positive charge in the same molecule. Examples of preferred zwitterionic surfactants are the betaines, the N-alkyl-N,N-dimethylammonium glycinates, the N-acyl-aminopropyl-N,N-dimethylammoniumglycinates and the 2-alkyl-3-carboxymethyl-3-hydroxyethyl-imidazolines each having 8 to about 24 carbon atoms in the alkyl group. Examples of preferred ampholytic surfactants are N-alkylglycines, N-alkylaminopropionic acids, N-alkylaminobutyric acids, N-alkyliminodipropionic acids, N-hydroxyethyl-N-alkylamidopropyl-glycines, N-alkyltaurines, N-alkylsarcosines, 2-alkylaminopropionic acids and alkylaminoacetic acids each having 8 to about 24 carbon atoms in the alkyl group.

The cosmetic agents as contemplated herein, which are formulated as an emulsion, in particular as an oil-in-water emulsion, preferably contain at least one nonionic oil-in-water emulsifier having an HLB value of more than about 7 to about 20. These are emulsifiers which are generally known to the person skilled in the art. For ethoxylated products, the HLB value is calculated according to the formula HLB=(100−L):5 wherein L is the weight fraction of the lipophilic groups, that is, the fatty alkyl or fatty acyl groups, in the ethylene oxide adducts, expressed in weight percent. In this context, it can be preferred as contemplated herein when, furthermore, a water-in-oil emulsifier having an HLB value of greater than about 1.0 and less than or equal to about 7.0 is used.

Preferably used for thickening the cosmetic agent as contemplated herein are hydrogel-forming substances which are selected from cellulose ethers, especially hydroxyalkylcelluloses, in particular hydroxypropylcellulose, hydroxypropylmethylcellulose, hydroxyethylcellulose, carboxymethylcellulose, cetylhydroxyethylcellulose, hydroxybutylmethylcellulose, methylhydroxyethylcellulose, furthermore xanthan gum, sclerotium gum, succinoglucans, polygalactomannans, in particular guar gum and locust bean gum, in particular guar gum and locust bean gum itself and the nonionic hydroxyalkyl guar derivatives and locust bean gum derivatives such as hydroxypropyl guar, carboxymethylhydroxypropyl guar, hydroxypropylmethyl guar, hydroxyethyl guar and carboxymethylguar, furthermore pectins, agar, carrageenan, tragacanth, gum arabic, karaya gum, tara gum, gellan, gelatin, casein, propylene glycol alginate, alginic acids and their salts, in particular sodium alginate, potassium alginate and calcium alginate, furthermore polyvinylpyrrolidones, polyvinyl alcohols, polyacrylamides. Particularly preferred hydrogel formers are selected from cellulose ethers, especially from hydroxyalkylcelluloses, in particular from hydroxypropylcellulose, hydroxypropylmethylcellulose, hydroxyethylcellulose, carboxymethylcellulose, cetylhydroxyethylcellulose, hydroxybutylmethylcellulose and methylhydroxyethylcellulose, as well as mixtures thereof. However, it is also possible to use a lipophilic thickening agent. Lipophilic thickening agents preferred as contemplated herein are selected from hydrophobized clay minerals and pyrogenic silicic acids.

The cosmetic agents as contemplated herein can additionally contain at least one skin-cooling active ingredient. Skin-cooling active ingredients suitable as contemplated herein are, for example, menthol, isopulegol and menthol derivatives, for example, menthyl lactate, menthyl glycolate, menthyl ethyl oxamate, menthyl pyrrolidone carboxylic acid, menthyl methyl ether, menthoxypropanediol, menthone glycerol acetal (9-methyl-6-(1-methylethyl)-1,4-dioxaspiro (4.5) decane-2-methanol), monomethyl succinate, 2-hydroxymethyl-3,5,5-trimethylcyclohexanol, and 5-methyl-2-(1-methylethyl) cyclohexyl-N-ethyloxamate. Preferred skin-cooling active ingredients are menthol, isopulegol, menthyl lactate, menthoxypropanediol, menthylpyrrolidonecarboxylic acid and 5-methyl-2-(1-methylethyl) cyclohexyl-N-ethyl oxamate, and mixtures of these substances, in particular mixtures of menthol and menthyl lactate, menthol, menthol glycolate and menthyl lactate, menthol and menthoxypropanediol or menthol and isopulegol.

Furthermore, it can be provided that the cosmetic agents as contemplated herein contain a propellant. In this case, they are formulated as a propellant-driven aerosol. Preferred propellants (propellant gases) are propane, propene, n-butane, isobutane, isobutene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropenes, both individually and in mixtures thereof. Hydrophilic propellant gases, such as carbon dioxide, can also be used advantageously in the context of the present disclosure, when the proportion of hydrophilic gases is selected low and lipophilic propellant gas (for example, propane/butane) is present in excess. Particularly preferred are propane, n-butane, isobutane, and mixtures of these propellant gases. It has been found that the use of n-butane as an only propellant gas as contemplated herein can be particularly preferred. The total amount of propellant is from about 20 to about 95% by weight, preferably from about 30 to about 85% by weight, in particular from about 40 to about 75% by weight, each based on the total weight of the cosmetic agent, including the above-described components a) and b) and optionally the additional active and inactive ingredients and the propellant.

The application of the cosmetic agent as contemplated herein can be carried out by employing various methods. According to a first embodiment, the cosmetic agent is formulated as a spray application. The spray application is carried out with a spray device which contains a filling of the liquid, viscous-flowable, suspension-formed or powdered antiperspirant cosmetic agent as contemplated herein in a container. The filling can be under the pressure of a propellant (compressed gas cans, compressed gas packages, aerosol dispensers), or it can be a mechanically operated pumping atomizer without propellant gas (pump sprays/squeeze bottle). The containers have a removal device, preferably in the form of valves, which enable the removal of the contents as a mist, smoke, foam, powder, paste or liquid jet. Cylindrical vessels made of metal (aluminum, tinplate, volume preferably maximum of about 1,000 ml), protected or non-splitting glass or plastic (preferably maximum of about 220 ml) or splintering glass or plastic (volume preferably from about 50 to about 400 ml) are especially considered as containers for the spraying devices. Creamy, gelatinous, pasty and liquid agents can be packaged, for example, in pump, spray or squeeze dispensers, in particular in multi-chamber pumping, multi-chamber spray or multi-chamber squeeze dispensers. The packaging for the agents as contemplated herein can be opaque, but also transparent or translucent.

According to a second embodiment, the cosmetic agent as contemplated herein can be formulated as a stick, soft solid, cream, roll-on, dibenzylidenalditol-based gel, loose or compact powder. The formulation of the cosmetic agents as contemplated herein in a specific administration form, such as an antiperspirant roll-on, an antiperspirant stick or an antiperspirant gel, is preferably based on the requirements of the intended use. Depending on the intended use, the cosmetic agents as contemplated herein can therefore be present in solid, semisolid, liquid, disperse, emulsified, suspended, gelatinous, multiphase or powdery form. In the context of the present disclosure, the term liquid also includes any types of solids dispersions in liquids. In the context of the present disclosure, multiphase cosmetic agents as contemplated herein are also understood to mean agents which have at least 2 different phases with a phase separation and in which the phases can be arranged horizontally, that is, one above the other, or vertically, that is, side by side.

The application can be performed, for example, using a roller ball applicator. Such rollers have a ball mounted in a ball bed which can be moved by movement over a surface. In this case, the ball takes up some of the cosmetic agent as contemplated herein to be distributed and conveys this to the surface to be treated. The packaging for these agents can, as stated previously, be opaque, transparent or translucent.

Furthermore, it is also possible to apply the cosmetic agents as contemplated herein by employing a solid stick in the form of a solid emulsion.

According to a third embodiment, the cosmetic agent as contemplated herein can be present on and/or in a disposable substrate selected from the group of wipes, pads and wads. Particularly preferred are wet wipes, that is, wet wipes prefabricated for the user, preferably individually packaged, as they are, for example, well known from the field of glass cleaning or from the field of wet toilet paper. Such wet wipes, which can advantageously also contain preservatives, are impregnated or applied with a cosmetic agent as contemplated herein and preferably individually packaged. They can, for example, be used as a deodorant cloth, which is particularly interesting for use on the go. Preferred substrate materials are selected from porous sheet-like cloths. They can be made of a fibrous or cellular flexible material which has sufficient mechanical stability and, at the same time, softness for application to the skin. These wipes include wipes made of woven and nonwoven synthetic and natural fibers, felt, paper or foam, such as hydrophilic polyurethane foam. Deodorizing or antiperspirant substrates which are preferred as contemplated herein can be obtained by saturating or impregnating or also by fusing a cosmetic agent as contemplated herein onto a substrate.

A second aspect of the present disclosure is a non-therapeutic cosmetic method for preventing and/or reducing the body odor triggered by transpiration and/or the transpiration of the body, in which a cosmetic agent as contemplated herein is applied to the skin, in particular to the skin of the armpits, and remains on the skin for at least about 1 hour, preferably for at least about 2 hours, more preferably for at least 4 hours, in particular for at least about 6 hours.

With regard to further preferred embodiments of the method as contemplated herein, in particular with regard to the cosmetic agents used there, what is said about the cosmetic agents as contemplated herein applies mutatis mutandis.

Finally, a further subject of the present disclosure is the use of a cosmetic agent as contemplated herein for preventing and/or reducing the body odor of the body triggered by transpiration.

With regard to further preferred embodiments of the use as contemplated herein, in particular with regard to the cosmetic agents used, what is said about the cosmetic agents as contemplated herein and the method as contemplated herein applies mutatis mutandis.

### Examples

The following compositions were prepared (all specification in % by weight):

| | V1 | V2 | V3 | V4 | E1 | E2 |
|---|---|---|---|---|---|---|
| Steareth-2 | 2 | | | | | |
| Steareth-21 | 2.5 | | | | | |
| 1,2-propanediol | 3 | | | | | |
| PPG-15 stearyl ether | 3 | | | | | |
| Phenoxyethanol | 1 | | | | | |
| EDTA | 0.1 | 0.1 | — | — | — | — |
| EDDS | — | — | — | 0.1 | 0.1 | 0.22 |
| Cetyl dimethyl | — | 0.18 | 0.18 | — | 0.18 | 0.18 |
| benzalkonium chloride | | | | | | |

| | |
|---|---|
| Perfume | 1 |
| Water | ad 100 |

The effect of the various compositions against S. hominis was tested, wherein a preculture was adjusted with medium to a germ density of 10⁴ cfu/ml and dispensing in 100 ml aliquots onto Erlenmeyer flasks. The deodorant formulations to be tested were given for this purpose.

The culture was then incubated at 100 rpm at 37° C. After 24 h, a germ count was determined by plating in comparison to an untreated control. The germ count reduction is indicated in log levels:

| Formulation | Germ number reduction log levels |
|---|---|
| V1 | 0.9 |
| V2 | 5.2 |
| V3 | 0.7 |
| V4 | 1.1 |
| E1 | 6.2 |
| E2 | 6.1 |

The table shows that EDTA or EDDS alone (V1 or V4) are just as ineffective as benzalkonium chloride alone (V3). The combination of complexing agent and benzalkonium chloride is more effective (V2, E1, E2), wherein EDDS results clearly outperform EDTA.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent comprising, in a cosmetically acceptable carrier:
a) ethylenediamine disuccinic acid and/or salts thereof;
b) at least one benzalkonium chloride of the formula (I) 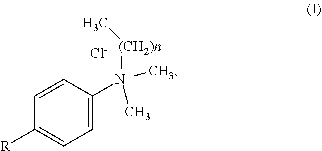
in which R stands for —H or -ethyl and n stands for an integer selected from the group of 7, 9, 11, 13, 15 or 17; and wherein
the total amount of compound(s) of formula (I) is from about 0.05 to about 5% by weight, based on a total weight of the cosmetic agent.

2. The cosmetic agent according to claim 1, wherein, the at least one benzalkonium chloride(s) of the formula (I) is present in the cosmetic agent in an amount of from about 0.05 to about 2.5% by weight, based on the total weight of the cosmetic agent.

3. The cosmetic agent according to claim 1, wherein the cosmetic agent comprises, based on its weight, from about 0.01 to about 1% by weight of the ethylenediamine disuccinic acid and/or salts thereof.

4. The cosmetic agent according to claim 1, wherein the total amount of ingredients a) and b), based on the total weight of the cosmetic agent, is from about 0.075 to about 2.5% by weight.

5. The cosmetic agent according to claim 1, wherein the weight ratio of the ingredients a) and b) to one another is from about 5:1 to about 1:5.

6. The cosmetic agent according to claim 1, wherein the cosmetic agent additionally comprises one or more active ingredients selected from the group of triethyl citrate, 3-[(2-ethylhexyl)oxy]-1,2-propanediol, 2-benzylheptanol, phenoxyethanol, cocamidopropyl PG-dimonium chloride phosphate, tropolone, silver lactate, magnesium oxide, aluminum chlorohydrate, aluminum-zirconium tetrachlorohydrexglycine and mixtures thereof.

7. The cosmetic agent according to claim 1, wherein the cosmetic agent additionally comprises one or more active ingredients selected from the group of cetylpyridinium chloride, zinc hydroxide carbonate, zinc phenolsulfonate, polyglyceryl-2-caprate, sorbitan caprylate, octenidine, charnesolic acid, tartaric acid and mixtures thereof.

8. The cosmetic agent according to claim 1, wherein the cosmetic agent is free of EDTA and/or its salts.

9. A method for preventing and/or reducing body odor, the method comprising the steps of:
applying a cosmetic agent to the skin, wherein the cosmetic agent comprises a) ethylenediamine disuccinic acid and/or salts thereof, and b) b) at least one benzalkonium chloride of the formula (I) 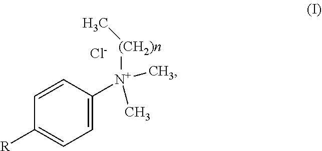
in which R stands for —H or -ethyl and n stands for an integer selected from the group of 7, 9, 11, 13, 15 or 17, and wherein the total amount of the compound(s) of formula (I) is from about 0.05 to about 5% by weight, based on a total weight of the cosmetic agent; and
leaving the cosmetic agent on the skin for at least 1 hour.

10. The cosmetic agent according to claim 1 wherein, the at least one benzalkonium chloride(s) of the formula (I) is present in the cosmetic agent in an amount of from about 0.15 to about 0.75% by weight, based on the total weight of the cosmetic agent.

11. The cosmetic agent according to claim 1 wherein, the at least one benzalkonium chloride(s) of the formula (I) is present in the cosmetic agent in an amount of from about 0.2 to about 0.5% by weight, based on the total weight of the cosmetic agent.

12. The cosmetic agent according to claim 1 wherein, the cosmetic agent comprises the ethylenediamine disuccinic acid and/or salts thereof in an amount of from about 0.03 to about 0.7% by weight, based on the total weight of the cosmetic agent.

13. The cosmetic agent according to claim 1 wherein, the cosmetic agent comprises the ethylenediamine disuccinic acid and/or salts thereof in an amount of from about 0.04 to about 0.6% by weight, based on the total weight of the cosmetic agent.

14. The cosmetic agent according to claim 1, wherein the total amount of ingredients a) and b) is from about 0.1 to about 2% by weight, based on the total weight of the cosmetic agent.

15. The cosmetic agent according to claim 1, wherein the total amount of ingredients a) and b) is from about 0.15 to about 1.0% by weight, based on the total weight of the cosmetic agent.

16. The cosmetic agent according to claim 1, wherein the weight ratio of the ingredients a) and b) to one another is from about 2:1 to about 1:2.

17. The cosmetic agent according to claim 1, wherein the weight ratio of the ingredients a) and b) to one another is from about 12:9 to about 1:1.

18. The cosmetic agent according to claim 6, wherein the additional one or more active ingredients are present in the cosmetic agent at from about 0.1 to about 10% by weight, based on the total weight of the cosmetic agent.

19. The cosmetic agent according to claim 1, wherein the cosmetic agent further comprises at least two active ingredients selected from the group of triethyl citrate, 3-[(2-ethylhexyl)oxy]-1,2-propanediol, 2-benzylheptanol, phenoxyethanol, cocamidopropyl PG-dimonium chloride phosphate, 1,2-hexanediol, 1,2-octanediol, 1,2-decanediol, tropolone, silver lactate, magnesium oxide, aluminum chlorohydrate, aluminum-zirconium tetrachlorohydrexglycine, and mixtures thereof.

20. The cosmetic agent according to claim 1, wherein the cosmetic agent further comprises at least two active ingredients selected from the group of cetylpyridinium chloride, zinc hydroxide carbonate, zinc phenolsulfonate, polyglyceryl-2-caprate, sorbitan caprylate, octenidine, charnesolic acid, tartaric acid, and mixtures thereof.
